(19) United States
(12) Patent Application Publication
(10) Pub. No.: US 2018/0361134 A1
(43) Pub. Date: Dec. 20, 2018

(54) DEVICE FOR INFECTION PREVENTION

(71) Applicant: CASE WESTERN RESERVE UNIVERSITY, Cleveland, OH (US)

(72) Inventors: James R. Rowbottom, Cleveland, OH (US); James D. Reynolds, Cleveland, OH (US); Tianbi Duan, Cleveland, OH (US); Edward F. Niedoba, Cleveland, OH (US); Nicolas Difranco, Cleveland, OH (US); Jackson Pilliod, Cleveland, OH (US)

(21) Appl. No.: 15/781,397

(22) PCT Filed: Dec. 5, 2016
(86) PCT No.: PCT/US16/65004

§ 371 Date: Jun. 4, 2018

Related U.S. Application Data
(60) Provisional application No. 62/262,628, filed on Dec. 3, 2015.

(51) Int. Cl. A61M 39/16; B08B 1/00; B08B 9/02; A61L 2/16; A61L 2/22

(52) U.S. Cl. CPC A61M 39/16; B08B 1/006; B08B 9/021; B65H 37/00; A61L 2/22; A61L 2202/24; A61L 2/16

(57) ABSTRACT

An apparatus includes sheet material having a first layer, a second layer extending generally parallel to the first layer, and at least one enclosure extending between the first and second layers. The enclosure is at least partially filled with an antimicrobial composition. A housing supports the sheet material for movement relative to the housing. The housing has an opening through which the sheet material extends to dispense the sheet material. In another aspect, a housing has a space storing an antimicrobial composition. The housing is connected to a desired object to be disinfected.

### RELATED APPLICATION

This application claims priority from U.S. Provisional Application No. 62/262,628, filed Dec. 3, 2015, the subject matter of which is incorporated herein by reference in its entirety.

### BACKGROUND OF THE INVENTION

The present invention is directed to an apparatus for use in preventing infection, and more specifically, to an apparatus for use in preventing intravenous injection port infection.

A known system for preventing intravenous injection port infection includes wiping an injection port with an alcohol swab.

### SUMMARY OF THE INVENTION

An apparatus includes sheet material having a first layer, a second layer extending generally parallel to the first layer, and at least one enclosure extending between the first and second layers. The enclosure is at least partially filled with an antimicrobial composition. A housing supports the sheet material for movement relative to the housing. The housing has an opening through which the sheet material extends to dispense the sheet material. In another aspect, a housing has a space storing an antimicrobial composition. The housing is connected to a desired object to be disinfected.

### BRIEF DESCRIPTION OF THE DRAWINGS

FIG. 1 is a pictorial illustration of an apparatus for use in preventing infection constructed in accordance with a first embodiment;

FIG. 2 is a front view of the apparatus of FIG. 1 with a portion removed to show an interior;

FIG. 3 is a schematic side view of a sheet material used in the apparatus of FIG. 1;

FIG. 4 is a schematic view of a second embodiment of the invention;

FIG. 5 is a pictorial view of another embodiment of the invention;

FIG. 6 is a schematic view of a portion of the apparatus of FIG. 5;

FIG. 7 is a pictorial illustration of another embodiment of the present invention;

FIG. 8 is a cross-sectional view of another embodiment of the present invention;

FIG. 9 is a pictorial illustration of another embodiment of the present invention; and

FIG. 10 is a pictorial illustration of another embodiment of the present invention.

### DESCRIPTION OF AN EMBODIMENT

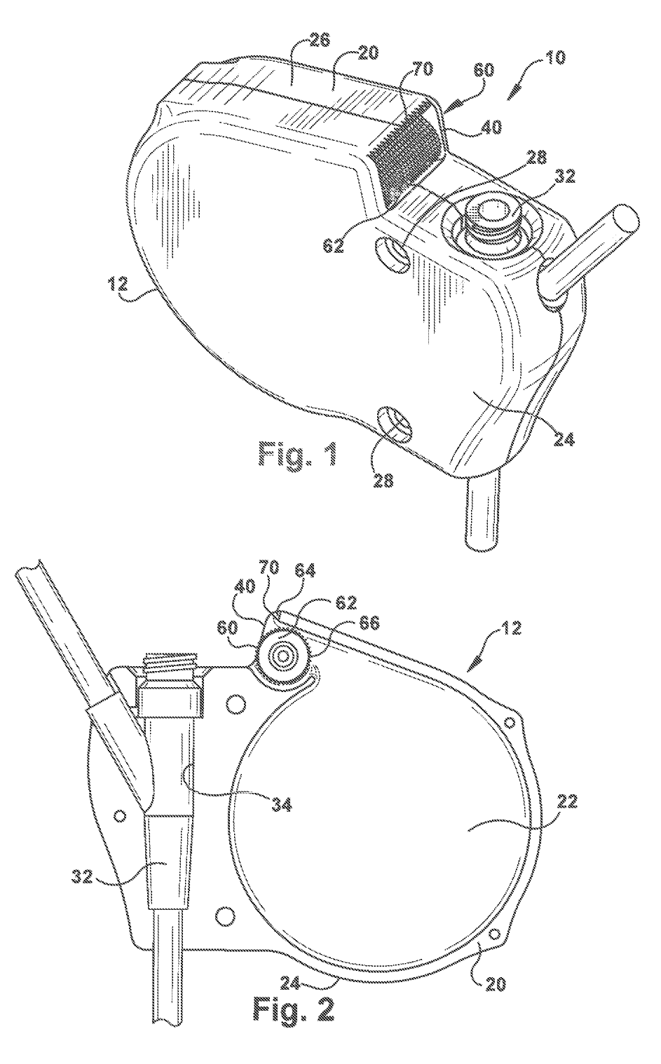

The present invention is directed to an apparatus 10 for use in preventing infection. The apparatus 10 includes a dispenser 12 (FIGS. 1 and 2) and an antimicrobial sheet material 14 (FIG. 3). The dispenser 12 (FIGS. 1 and 2) has a housing 20 with a space 22 for storing a roll of the sheet material 14. An end of the sheet material 14 may be removed from the housing 20 for disinfecting an object by unrolling the sheet material.

The housing 20 may have first and second portions 24, 26 connected together by fasteners 28, such as screws. Alternatively, the first and second portions 24, 26 may have a snap connection. The first and second portions 24, 26 define the space 22 for storing the roll of sheet material 14.

The dispenser 12 may be connected to any desired object to be disinfected, such as an intravenous injection port 32 or a multi-use drug bottle. The injection port 32 may extend through the housing 20. Each of the first and second portions 24, 26 may include a cavity 34 for receiving the injection port 32. The fasteners 28 may clamp the dispenser 12 to the injection port 32. It is also contemplated that the first and second portions 24, 26 may be connected to each other by a hinge. Alternatively, the dispenser 12 may include a clip for connecting the dispenser to the injection port 32. Also, the injection port 32 and housing 20 may be formed as one piece.

The housing 20 supports the roll of sheet material 14 for rotation in an unwinding direction. An end of the roll of sheet material 14 extends from an opening 40 in the housing 20 so that the end of the roll of sheet material may be manually grasped and pulled out of the housing. The opening 40 in the housing 20 may be located adjacent the intravenous injection port 32 when the dispenser 12 is connected to the injection port. The housing 20 may include a cylindrical projection extending into the space 22 and into the roll of sheet material 14 to rotatably support the sheet material for unwinding relative to the housing.

The sheet material 14 (FIG. 3) includes a first or film layer 44 with enclosures or bubbles 46 formed on the film layer. The film layer 44 and bubbles 46 may be made of any desired material, such as polyethylene. The bubbles 46 are at least partially filled with an antimicrobial composition 48. The antimicrobial composition 48 may contain isopropyl alcohol. The bubbles 46 may be filled by injecting the bubbles with the antimicrobial composition 48 and sealing the hole formed in the bubble. It is also contemplated that the bubbles 46 may include a resealable barrier, such as a silicone adhesive coating, that reseals after the bubble is injected with the antimicrobial composition.

The bubbles 46 are connected to a second or absorbent fabric layer 50 with the bubbles extending between the film layer 44 and the fabric layer. The bubbles 46 may be connected to the fabric layer 50 by a biocompatible adhesive. The fabric layer 50 may be made of any desired absorbent material, such as spunwoven polyester. Upon opening, rupturing, breaking and/or popping of a bubble 46, the fabric layer 50 absorbs the antimicrobial composition 48. The portion of the fabric layer 50 that absorbs the composition 48 may be used to disinfect the injection port 32 or other desired object to help prevent infection of a patient.

The dispenser 12 (FIG. 2) may include an enclosure or bubble opening device 60 that opens, ruptures, breaks or pops enclosures 46 on the sheet material 14 as the sheet material is pulled out of the dispenser. The enclosure opening device 60 may include a roller 62 in the housing 20. The sheet material 14 extends between the roller 62 and an edge 64 of the housing 20 defining the opening 40. The fabric layer 50 engages the roller 62. The roller 62 is spaced from the edge 64 a distance that is smaller than the bubbles 46 extend between the film layer 44 and the fabric layer 50. Therefore, the roller 62 applies a force to the enclosures 46 to open, pop, break or rupture the enclosures or bubbles as they pass between the roller and the edge 64. The roller 62 may have teeth 66 that grip the fabric layer 50 as the sheet material is pulled through the opening 40 so that the roller 62 rotates relative to the housing 20.

The edge 64 of the housing 20 may include a cutter 70 for cutting the sheet material 14 after the bubble 46 ruptures. The portion of the sheet material 14 with the ruptured bubble 46 and fabric layer 50 that absorbed the antimicrobial composition may be removed from the roll and used to disinfect the injection port 32. The sheet material 14 may include perforations between adjacent enclosures 46 to aide in removal of a ruptured enclosure. The portion of the sheet material 14 used to disinfect the injection port 32 may be disposed of after use.

It is contemplated that the sheet material 14 may include an adhesive for attaching the used portion of the sheet material to an intravenous stand or a syringe to indicate that the injection port 32 was disinfected. Also, the dispenser may include a counter that indicates how many enclosures have been used. The sheet material 14 may be bar coded and scanned by the counter. Although the sheet material 14 is described as being manually pulled from the housing 20, the dispenser 12 may include a motor for automatically feeding the sheet material from the dispenser.

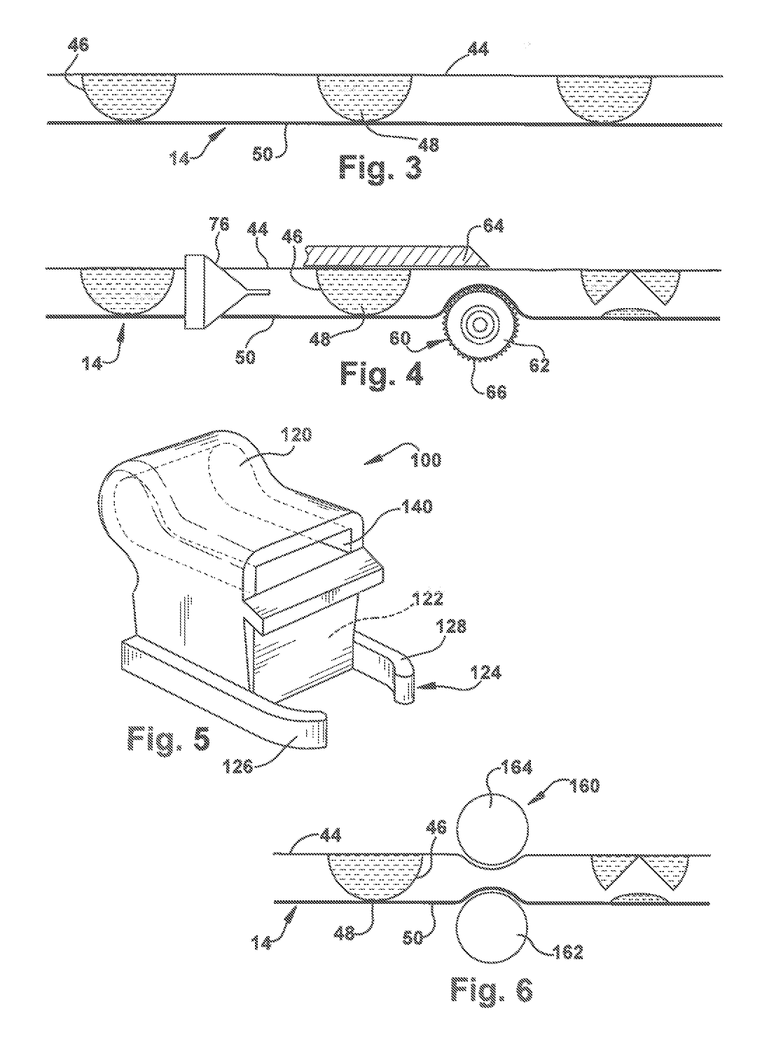

The dispenser 12 may include a valve 76 (FIG. 4) for preventing the antimicrobial composition 48 from entering the space 22 in the housing 20 after a bubble 46 ruptures. The valve 76 may be a duckbill check valve, as schematically shown in FIG. 4, through which the sheet material 14 passes. The enclosure 46 ruptures after passing through the valve 76 and the valve prevents the antimicrobial composition 48 from entering the space 22 and/or being absorbed by a portion of the fabric layer 50 upstream of the valve. The valve 76 may be connected to the housing 20 upstream of the enclosure opening device 60.

A dispenser 100 constructed in accordance with another embodiment of the present invention is illustrated in FIG. 5. The dispenser 100 has a housing 120 with a space 122 for storing a roll of the sheet material 14. The sheet material 14 may be removed from the housing 120 for disinfecting an object.

The housing 120 may be connected to any desired object to be disinfected, such as an intravenous injection port. The dispenser 100 may include a clip 124 for connecting the dispenser to the injection port. The clip 124 may include first and second arms 126, 128 extending generally parallel to each other from the housing 120. The arms 126, 128 may move away from each other as the injection port moves between the arms. The arms 126, 128 move toward each other to snap the housing 120 to the injection portion once the injection port moves between the arms to connect the housing to the injection port.

The housing 120 supports the roll of sheet material 14 for rotation in an unwinding direction. An end of the roll of sheet material 14 extends from an opening 140 in the housing 120. The end of the roll of sheet material 14 may be manually grasped and pulled out of the housing. The opening 140 in the housing 120 may be located adjacent the intravenous injection port when the dispenser 100 is connected to the injection port.

The dispenser 100 may include an enclosure or bubble opening device 160 (FIG. 6) that opens, ruptures, breaks or pops enclosures 46 on the sheet material 14 as the sheet material is pulled out of the dispenser. The enclosure opening device 160 may include a pair of rollers 162, 164 in the housing 120. The sheet material 14 extends between the rollers 162, 164. The rollers 162, 164 are spaced from each other a distance that is smaller than the bubbles 46 extend between the film layer 44 and the fabric layer 50. Therefore, the rollers 162, 164 apply a force to the enclosures 46 to open, pop, break or rupture the enclosures or bubbles as they pass between the rollers. Upon opening, rupturing, breaking and/or popping of a bubble 46, fabric layer 50 absorbs the antimicrobial composition 48. The portion of the fabric layer 50 that absorbs the composition 48 may be used to disinfect the injection port 32 or other desired object to help prevent infection of a patient.

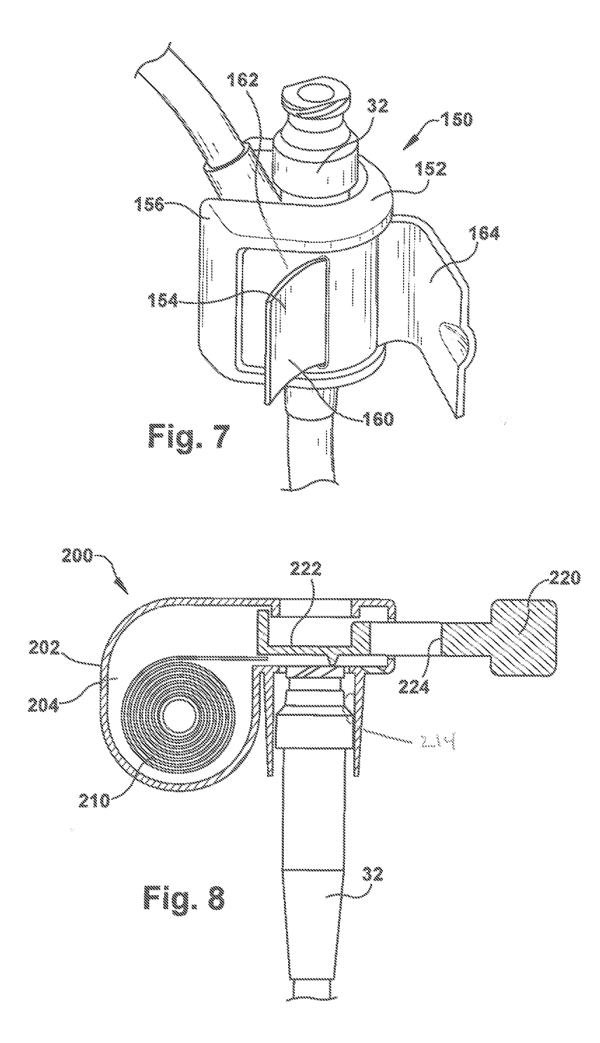

A dispenser 150 constructed in accordance with another embodiment of the present invention is illustrated in FIG. 7. The dispenser 150 has a housing 152 with a space for storing a sheet material 154. The sheet material 154 may be removed from the housing 152 for disinfecting an object. The housing 150 may be connected to any desired object to be disinfected, such as an intravenous injection port 32. The dispenser 150 may include a clip 156 for connecting the dispenser to the injection port 32.

The housing 152 may store a plurality of individual wipes 160. The wipes 160 may be impregnated with an antimicrobial composition. An individual wipe 160 may extend through an opening in the housing 152. The individual wipe 160 may be manually grasped and pulled out of the housing 152 for use in disinfecting the injection port 32. The housing 152 may have a recess 162 for storing an end of the wipe 160 extending from the housing. A closure member or door 164 may be pivotally connected to the housing 152. The closure member 164 may keep the wipe 160 in the recess 162 when in a closed position to prevent the wipe from drying out. The door 164 may be pivoted from the closed position to an open position, shown in FIG. 7, to allow the wipe 160 to be grasped.

A dispenser 200 constructed in accordance with yet another embodiment of the present invention is illustrated in FIG. 8. The dispenser 200 has a housing 202 with a space 204 for storing a roll of sheet material 210. The sheet material 210 may include individual antimicrobial pads. The pads may be treated with an antimicrobial composition.

The housing 202 has an opening 214 that receives an object to be disinfected, such as an injection port 32. A cover member 220 extends generally perpendicular to the opening 214. The cover member 220 moves relative to the housing in a direction generally perpendicular to an axis of the opening 214. The cover member 220 has a cover portion 222 and an opening 224. The cover portion 222 closes the opening 214 in the housing 202 when the cover member 220 is in a first position, shown in FIG. 8. The opening 224 in the cover member 220 is aligned with the opening 214 in the housing 202 when the cover member is in a second position. Therefore, the injection port 32 may be accessed by a syringe when the cover member is in the second position.

The cover member 220 moves from the first position to the second position to permit access to the injection port 32. A syringe may extend into the opening 214 in the housing 202. The syringe also extends through the opening 224 in the cover member and the antimicrobial pad on the sheet material 202 to disinfect the injection port 32. The cover member 220 then moves from the second position to the first position. A projection on the cover member 220 pulls the sheet material 210 to unwind the sheet material and position a new antimicrobial pad over the top of the injection port. The movement of the sheet material 210 also moves the used antimicrobial pad out of the housing 202 for disposal. The cover member 220 then moves from the first position back to the second position to permit another syringe to be inserted into the injection port.

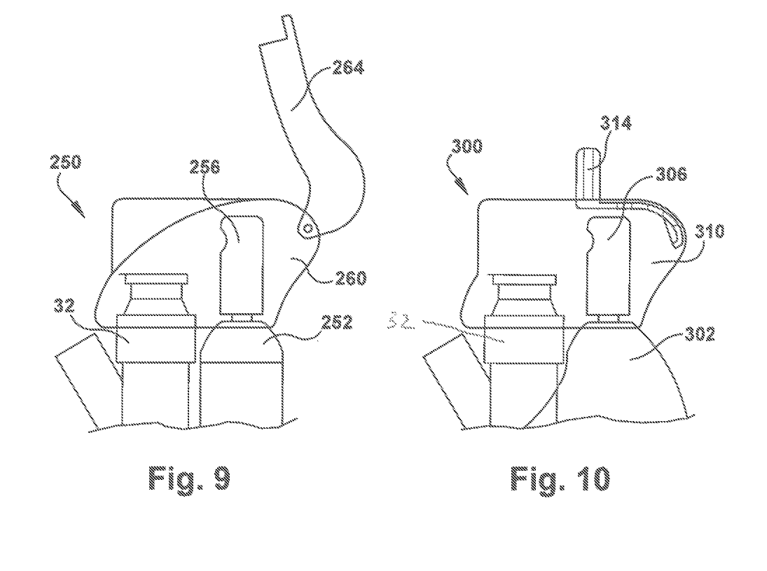

A dispenser 250 constructed in accordance with yet another embodiment of the present invention is illustrated in FIG. 9. The dispenser 250 has a housing 252 with a space for storing an antimicrobial composition. The antimicrobial composition may be dispensed from the space by a spray nozzle 256. The housing 252 may be connected to an object to be disinfected, such as an injection port 32.

The nozzle 256 extends into an enclosure 260 adjacent to the injection port 32. The nozzle 256 sprays the antimicrobial composition onto the injection port 32 when depressed. A closure member 264 is pivotally connected to the enclosure 260. The closure member 264 has a first or open position, shown in FIG. 9 and a second or closed position. A syringe may be inserted into the injection port 32 when the closure member 264 is in the first position. The closure member 264 engages the spray nozzle 256 when in the second position. The closure member 264 may be pressed to depress the spray nozzle 256. Therefore, the closure member 264 is pressed when in the closed position to depress the nozzle 256 and spray antimicrobial composition onto the injection port 32 to disinfect the injection port. The closure member 264 moves to the open position and a syringe is inserted into the injection port 32 after the nozzle 256 has been depressed.

A dispenser 300 constructed in accordance with yet another embodiment of the present invention is illustrated in FIG. 10. The dispenser 300 has a housing 302 with a space for storing an antimicrobial composition. The antimicrobial composition may be dispensed from the space by a spray nozzle 306. The housing 302 may be connected to an object to be disinfected, such as an injection port 32.

The nozzle 306 extends into an enclosure 310 adjacent to the injection port 32. The nozzle 306 sprays the antimicrobial composition onto the injection port 32 when depressed. A closure member 314 is slidably connected to the enclosure 310. The closure member 314 has a first or open position, shown in FIG. 10 and a second or closed position. A syringe may be inserted into the injection port 32 when the closure member 314 is in the first position. The closure member 314 engages the spray nozzle 306 when in the second position. The closure member 314 may be slid from the first position to the second position to depress the spray nozzle 306. Therefore, the closure member 314 slides from the open position to the closed position to depress the nozzle 306 and spray antimicrobial composition onto the injection port 32 to disinfect the injection port. The closure member 314 moves to the open position and a syringe is inserted into the injection port 32 after the nozzle 306 has been depressed.

The apparatus may be used to administer antimicrobial wipes or strips at any desired device and, more specifically, at an intravenous injection port.

From the above description of the invention, those skilled in the art will perceive improvements, changes and modifications. Such improvements, changes and modifications within the skill of the art are intended to be covered by the appended claims.

Having described the invention, the following is claimed:

1. An apparatus comprising:
sheet material having a first layer, a second layer extending generally parallel to the first layer, and at least one enclosure extending between the first and second layers, the enclosure being at least partially filled with an antimicrobial composition; and
a housing supporting the sheet material for movement relative to the housing, the housing having an opening through which the sheet material extends to dispense the sheet material.

2. An apparatus as set forth in claim 1 wherein the second layer is an absorbent layer that absorbs the antimicrobial composition upon opening the enclosure.

3. An apparatus as set forth in claim 2 wherein the first and second layers extend generally parallel to each other and a plurality of enclosures extend between the layers, the plurality of enclosures being spaced apart along a centerline of the first layer.

4. An apparatus as set forth in claim 1 further including an enclosure opening device that opens the enclosure as the sheet material is removed from the housing.

5. An apparatus as set forth in claim 4 wherein the enclosure opening device includes at least one roller supported by the housing that applies a force to the enclosure to cause the enclosure to open.

6. An apparatus as set forth in claim 4 wherein the at least one roller is spaced from the housing a distance smaller than the enclosures extend between the first and second layers.

7. An apparatus as set forth in claim 4 wherein the enclosure opening device includes first and second rollers supported by the housing, the first and second rollers being spaced from each other a distance smaller than the enclosures extend between the first and second layers.

8. An apparatus as set forth in claim 4 wherein the at least one roller includes teeth engaging one of the first and second layers.

9. An apparatus as set forth in claim 1 wherein the housing includes a cutter for cutting the sheet material.

10. An apparatus as set forth in claim 1 wherein the housing is connected to a desired object to be disinfected.

11. An apparatus as set forth in claim 10 wherein the housing includes first and second portions connected together, the first and second portions including a cavity for receiving the desired object.

12. An apparatus as set forth in claim 11 wherein the housing includes a clip for connecting the housing to the desired object.

13. An apparatus as set forth in claim 1 wherein the sheet material extends through a valve that prevents the antimicrobial composition from entering a space in the housing for a roll of sheet material after an enclosure opens.

14. An apparatus as set forth in claim 13 wherein the valve is a duckbill check valve.

15. An apparatus as set forth in claim 1 wherein the first layer is a film layer and the second layer is an absorbent layer, the enclosures being formed on the first layer and connected to the second layer.

16. An apparatus comprising:
a housing having a space storing an antimicrobial composition, the housing being connected to a desired object to be disinfected.

17. An apparatus as set forth in claim 16 wherein the housing includes first and second portions connected together, the first and second portions including a cavity for receiving the desired object.

18. An apparatus as set forth in claim 16 wherein the housing includes a clip for connecting the housing to the desired object.

19. An apparatus as set forth in claim 16 wherein the housing includes an opening that receives the desired object.

20. An apparatus as set forth in claim 16 wherein the housing supports sheet material for movement relative to the housing, the housing having an opening through which the sheet material extends to dispense the sheet material.

21. An apparatus as set forth in claim 20 wherein the sheet material has a first layer, a second layer extending generally parallel to the first layer, and an enclosure extending between the first and second layers, the enclosure being at least partially filled with the antimicrobial composition.

22. An apparatus as set forth in claim 16 wherein a closure member is pivotally connected to the housing, the closure member covering a wipe impregnated with the antimicrobial composition in a recess in the housing to prevent the wipe from drying out.

23. An apparatus as set forth in claim 16 further including a cover member having a cover portion and an opening, the cover member being movable relative to the housing between first and second positions, the cover portion closing an opening in the housing when the cover member is in the first position, the opening in the cover member being aligned with the opening in the housing when the cover member is in the second position.

24. An apparatus as set forth in claim 23 wherein the cover member moves a sheet material impregnated with the antimicrobial composition relative to the housing as the cover member moves from the second position to the first position.

25. An apparatus as set forth in claim 16 further including a spray nozzle extending into an enclosure, the nozzle spraying the antimicrobial composition when depressed, a closure member movably connected to the enclosure has a first position and a second position, the closure member engaging the spray nozzle when in the second position.
